(19) United States
(12) Patent Application Publication
(10) Pub. No.: US 2010/0322963 A1
(43) Pub. Date: Dec. 23, 2010

(54) LOW DOSE INOCULATION WITH TAP FOR ANTI-TUMOR IMMUNITY

(75) Inventors: WILFRED A. JEFFERIES, Surrey (CA); YUANMEI LOU, Vancouver (CA); ROBYN P. SEIPP, Vancouver (CA); SUSAN SHU-PING, Vancouver (CA); TIMOTHY Z. VITALIS, Vancouver (CA); TERRY W. PEARSON, Victoria (CA)

Correspondence Address: RISSMAN HENDRICKS & OLIVERIO, LLP, 100 Cambridge Street, Suite 2101, BOSTON, MA, 02114, US

(73) Assignee: Tapimmune, Inc., Vancouver (CA)

(21) Appl. No.: 12/487,019

(22) Filed: Jun. 18, 2009

(51) Int. Cl. A61K 39/00; A61P 35/00

(52) U.S. Cl. 424/199.1

(57) ABSTRACT

A safe and efficacious method of inducing protective immunity in animals against TAP-1 deficient metastatic melanoma is disclosed. The method involves treating the animals with relatively small amounts of a recombinant non-replicating adenovirus encoding human TAP-1 to promote and maintain long term anti-tumor survival, and enhanced memory T-cell subpopulations, even when the treatment effects only a small fraction of metastatic tumor cells.

### BACKGROUND OF THE INVENTION

The goal of anti-tumor vaccines is to elicit protective and therapeutic immune responses against highly autologous tumors. The basic problem with the majority of vaccines, immunotherapies and gene therapy approaches against tumors is that many metastatic tumor types appear to be immunoselected to lose the ability to present neoantigens to effector T cells, thereby subverting the immunosurveillance mechanisms that are thought to limit the emergence of malignant cells. Of primary importance is the loss of MHC I molecules at the cell surface, a phenomenon that is largely rooted in the down-regulation of components of the antigen processing machinery that normally allows MHC I loaded with the appropriate neoantigen to appear at the cell surface. As a result of this, in most strategies, every tumor cell needs to be transduced by the delivery vehicle for efficacious treatment of the disease. This does not appear possible, and certainly not when non-replicating viruses are used as the delivery vehicle. This approach is further limited in widely disseminated tumors that are distributed throughout the body, as increased toxicity is associated with increasing the number of non-replicating, recombinant viral particles. Thus, in their present form the number of delivered particles cannot approximate the total number of normal cells and tumor cells in the body, and therefore it is not possible to ensure that each tumor cell is transduced.

Although these problems have not been solved in the cancer vaccine field in general, they must ultimately be overcome for useful therapeutic vaccines to emerge. In addition, it would be highly desirable that such responses involve multiple T cell clones specific for multiple tumor antigens. Known tumor-associated antigens (TAAs) include non-mutated, over-expressed, or inappropriately expressed differentiation antigens. For example, in melanomas, antigens such as gp100, MART-1, TRP-1, TRP-2, and tyrosinase represent one class of tumor antigens that share expression with normal melanocytes, the cell of origin of this cancer. Immunization against these normal, non-mutated melanoma/melanocyte antigens represents a unique challenge because of potential T cell tolerance or anergy that may inhibit immune reactivity against normal self-tissues. Much preclinical and clinical data have been generated using a variety of cancer vaccines involving peptides, naked DNA or RNA encoding TAAs, recombinant viruses encoding TAAs, whole tumor cells, dendritic cells and heat shock proteins. However, these current approaches have not been effective in mediating cancer regression.

The most effective immunotherapy method to date in both mice and patients with metastatic cancer involves the adoptive transfer of anti-tumor lymphocytes into lympho-depleted hosts. Using this approach, tumor regression can be seen in mice or patients with metastatic melanomas that are refractory to other treatments. However, passive transfer of T cells is not expected to yield the long-lived tumor-specific immunity that might be required to prevent tumor progression or relapse. Ideal cancer vaccines should induce both tumor-specific effector T cells capable of reducing and/or eliminating the tumor mass, and the long-lasting tumor-specific memory T cells capable of controlling tumor relapse.

Dendritic cells are increasingly used as adjuvants for vaccination against cancer due to their capacity to induce tumor-specific cytotoxic (killer) and helper T cells. Many experiments in animals and human clinical trials suggest that dendritic cell vaccination has the potential to induce immune responses to cancer and might have considerable therapeutic potential.

Despite continued progress towards understanding the pathophysiology of tumor progression and metastasis, curative therapeutic options are still missing for metastatic melanoma. Downregulation of MHC class I antigen expression is frequently associated with impaired transporter-associated-with-antigen-processing (TAP) expression in many tumors, including melanoma, making them invisible to effector cytotoxic T cells.

One approach to making therapeutic vaccines is to use genetically engineered non-replicating viruses as vaccine vehicles to revive immunosurveillance mechanisms in order for tumors to be eradicated. A perceived problem with this approach is that the number of nonreplicating viruses used as vaccine inoculum does not remotely approximate the total number of cells in the body, nor even the number of tumor cells in the case of large tumor burden or metastasis, leaving some to argue that this is a poor method of anti-cancer vaccination.

Accordingly, it is an objective of the present invention to provide an improved method for immunizing a patient against metastatic melanoma employing genetically engineered non-replicating viruses as vaccine vehicles using a safe and efficacious treatment protocol.

### SUMMARY OF THE INVENTION

The present invention, in one aspect, is based on the discovery that a limited amount of vaccine inoculum of recombinant adenovirus encoding human TAP-1 can induce a protective immunity against TAP-deficient metastatic melanoma cells. The amount of recombinant cells required to achieve this result is on the order of 1×10⁸ cells per inoculum, or in the range of from about 1×10⁹ to 1×10⁷ cells per inoculum. Accordingly, efficacious anti-tumor responses are induced by injecting melanoma-bearing animals with relatively small amounts of recombinant viral cells, resulting in increased animal survival and in enhanced memory T cell subpopulations.

The inclusion of TAP in virus vaccines has been found to promote and maintain anti-tumor responses even when the vector used to deliver TAP only infects a small fraction of the metastatic tumor cells. This novel approach uses a limited amount of inoculate relative to the tumor cell mass, and thus achieves an efficacious outcome that has so far eluded other vaccine, immunotherapeutic or gene therapeutic strategies where there is a requisite for the majority of tumor cells to be transduced for beneficial outcomes to be achieved.

### BRIEF DESCRIPTION OF THE DRAWINGS

FIG. 1A is an immunoblot showing that hTAP-1 expression after infection by AdhTAP-1 leads to increased endogenous mTpn expression. Murine B16F10 melanoma cells were infected with AdhTAP-1 or Ψ5 50 pfu/cell and harvested 48 hrs later. The infected cells were analyzed for hTAP-1, mTAP-1, mTAP-2, and mTpn expression by immunoblotting. β-actin was used as a control for protein loading. T-1 and T-2 cells, respectively, were used as positive and negative controls for hTAP-1 expression. IFN-γ treated B16F10 cells and untreated B16F10 cells, respectively, were used as positive and negative controls for mTAP-1, mTAP-1 and mTpn expression.

FIG. 1B are bar graphs showing that Figure AdhTAP-1 infection increases surface MHC Class I expression. (A) H-2K^{b} and (B) H-2D^{b} surface expression in B16F10 cells was assessed by flow cytometric analysis. B16F10 cells were infected with AdhTAP-1 at or Ψ5 at 50 pfu/cell. Ψ5-adenovirus vector alone (negative control) and IFN-γ (positive control).

FIG. 1C is a graph showing the infection of B16F10 cells with AdhTAP-1 (50 pfu/cell) restores MHC class I antigen presentation of the TRP-2 epitope and increases susceptibility to lysis by TRP-2 specific effector cells. Splenocytes from mice immunized with TRP-2 peptide followed by irradiated RMA-S cells pulsed with TRP-2 were used as effectors. Targets: B16F10, B16F10 infected with Ψ5 (adenovirus vector control) or B16F10 infected with AdhTAP-1.

FIG. 1D is a bar graph showing TAP-1 expression in B16F10 cells increases the numbers of tumor-specific, IFN-γ secreting splenocytes. Bars represent the mean number of IFN-γ secreting splenocytes isolated from mice immunized with γ-irradiated B16F10 cells infected ex vivo with AdhTAP-1, Ψ′5 (Adenovirus vector control) or no treatment (PBS). Splenocytes from immunized mice were stimulated with the tumor associated antigens TRP-2 or gp100 or incubated without peptide. The numbers of tumor antigen-specific, IFN-γ secreting precursors were determined by ELISPOT assay. Precursor frequency is reported as IFN-γ-secreting cells per 10⁶ splenocytes (IFN-γ SC/10⁶ splenocytes).

FIG. 2 shows that AdhTAP-1 treatment retards tumor growth in mice bearing B16F10 tumors. C57BL/6 mice were injected s.c. with 1.5×10⁵ B16F10 cells/mouse and 1, 4, and 8 days after B16F10 cells were introduced, mice were treated sc with 108 pfu/mouse of AdhTAP-1 or Ψ5 or PBS only. AdhTAP-1 significantly retarded tumor growth of B16F10-bearing mice (p<0.01) compared to the Ψ5 and PBS-treated mice. Bars in FIGS. 2A and 2B indicate the mean for each group (12 mice per group). FIG. 2C is an immunoblot for AdhTAP-1, Ψ5 and PBS.

FIG. 3 is a series of photomicrographs showing that tumor infiltrating lymphocytes and dendritic cells (“DCs”) are increased in B16F10 tumors treated with AdhTAP-1. (A) Immunohistochemical staining for CD4⁺ (A, D, G), CD8⁺ (B, E, H) or CD11c⁺ (C, F, I) cells in B16F10 tumors treated with AdhTAP-1 (A, B, C), Ψ5 (Ad vector control) (D, E, F), or PBS (G, H, I) (200× magnification). Tumors were analyzed 15 days after B16F10 cells were introduced into the mice. A positive stain is indicated by the intense brown labeling of cell surface membranes. (B) Flow cytometric analysis shows that increased numbers of CD8⁺ T cells were present in B16F10 tumors treated with AdhTAP-1 in vivo. Tumors from mice treated with vector alone and AdhTAP-1 showed increased CD4⁺ T cell levels compared to PBS-treated mice. CD4⁺ and CD8⁺ T cell numbers are presented as a percentage of the viable tumor-derived cells isolated using Ficoll-Paque™ PLUS.

FIG. 4 is a series of graphs showing AdhTAP-1 infection increases surface MHC Class I expression in the human melanoma cell line buf1280. (A) HLA-ABC surface expression in buf1280 cells and (B) H-2K^{b} surface expression in buf1280/K^{b} cells determined by flow cytometric analysis. Buf1280 and buf1280K^{b} cells were infected with AdhTAP-1 at or Ψ5 at 3 pfu/cell. Ψ5—(adenovirus vector control), buf1280 TAP-1 and buf1280 TAP-1/K^{b} cells—(positive control).

### DESCRIPTION OF THE PREFERRED EMBODIMENTS

The realization that human cancers express tumor associated antigens (“TAAs”) has stimulated research into the development of immunotherapies to mediate the regression of established tumors. Both prophylactic and therapeutic vaccines targeting a wide variety of cancers are being developed in several laboratories, worldwide. Four criteria are required for the immunologically mediated destruction of established tumors: first, sufficient numbers of immune cells with highly avid receptors for tumor antigens must be generated in vivo. Second, these cells must traffic to and infiltrate the tumor stroma. Third the immune cells must be activated at the tumor site to manifest appropriate effector mechanisms such as direct lysis or cytokine secretion capable of causing tumor destruction. Fourth, the tumors themselves must have sufficient antigen processing and presenting capability to present neoantigens on MHC I molecules to the stimulated T cells.

Great progress has been made in the field of cancer vaccination in the past decade. However, certain classes of tumors lacking components of the antigen presenting machinery may fail to be recognized by immune cells, even when anti-tumor immune responses are mounted in vivo, because such tumor cells are unable to present TAA on their surfaces. In order to revive the immunosurveillance mechanisms, most approaches require that the therapeutic neoantigen or gene construct be introduced into every tumor cell. At the moment this does not seem possible. However, the current approach based on the use of AdhTAP-1 to induce responses against TAAs potentially offers a way around this impasse: 1) it is applicable to many patients regardless of HLA type since expression of TAP-1 is not MHC-restricted; 2) it has the potential for inducing immune responses to multiple tumor antigens, including known and unknown TAAs, and may thus provide an advantage over antigen-specific treatments, since it would minimize the escape of tumors that present unknown TAAs; 3) it induces TAP-dependent cross-priming; 4) AdhTAP-1 infection would enhance endogenous tapasin expression in TAP- and tapasin-deficient tumors, and 5) it results in enhanced memory CD4⁺ and CD8⁺ T cell production. The latter two points are important for sustained anti-tumor immunity.

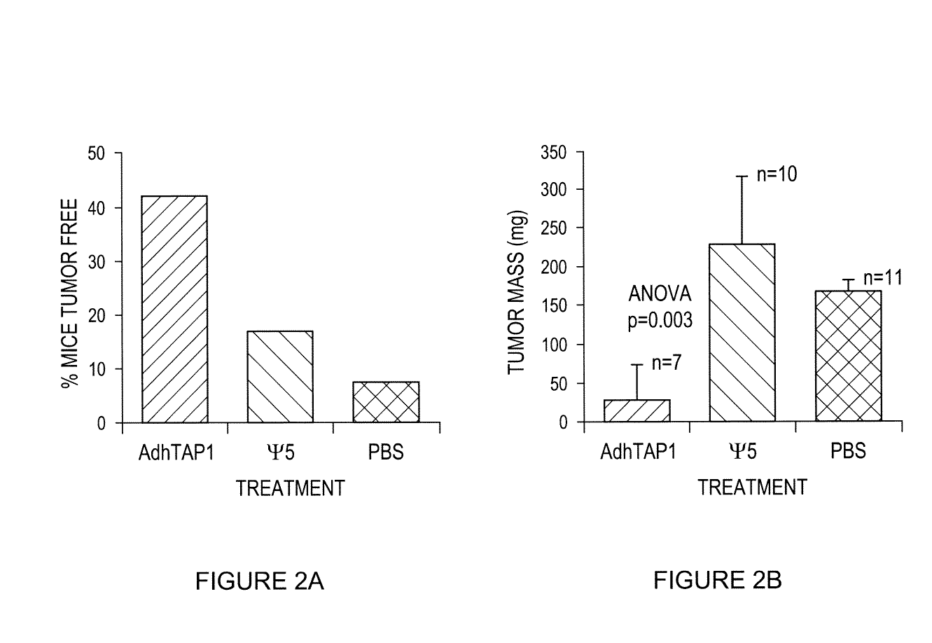

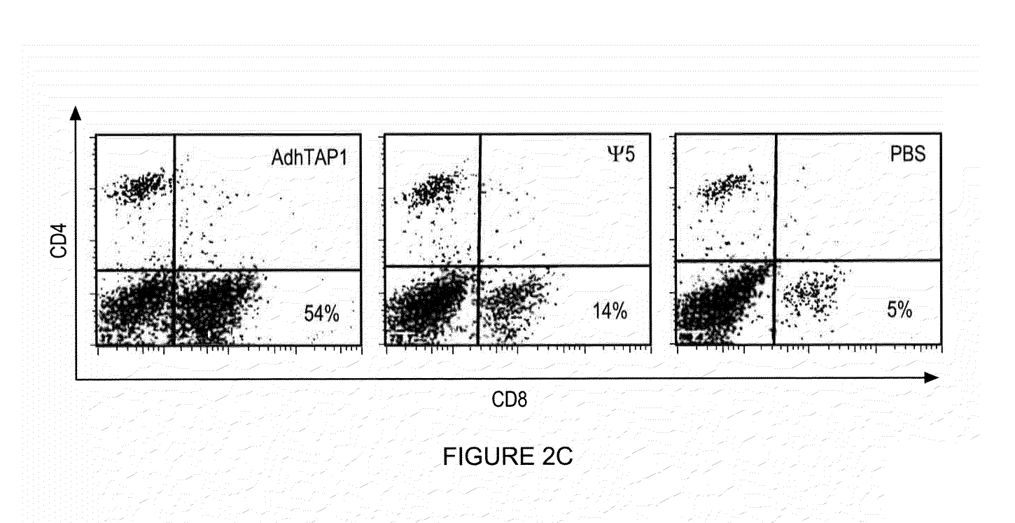

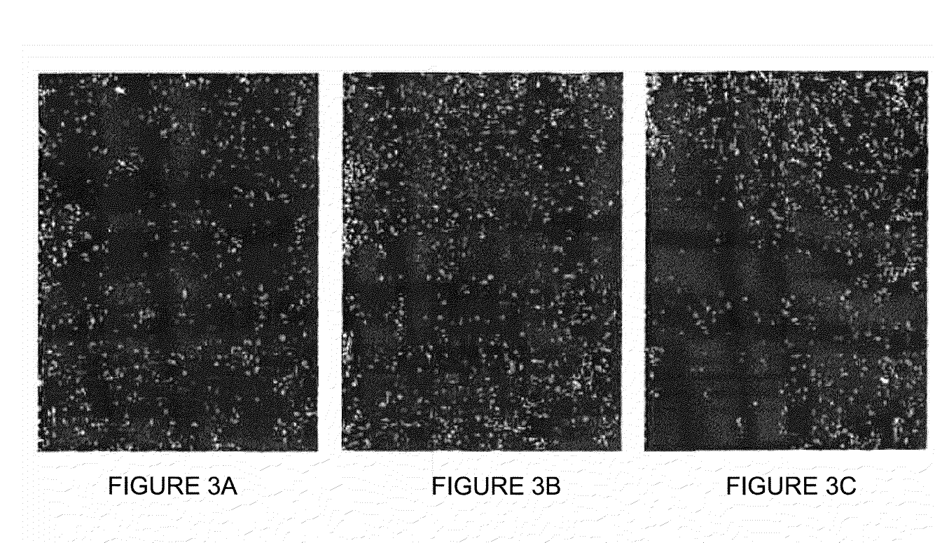

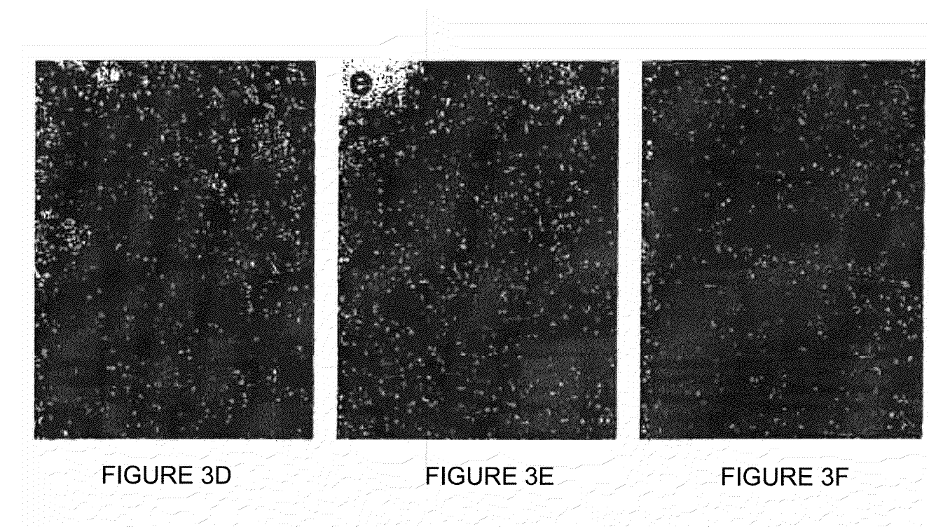

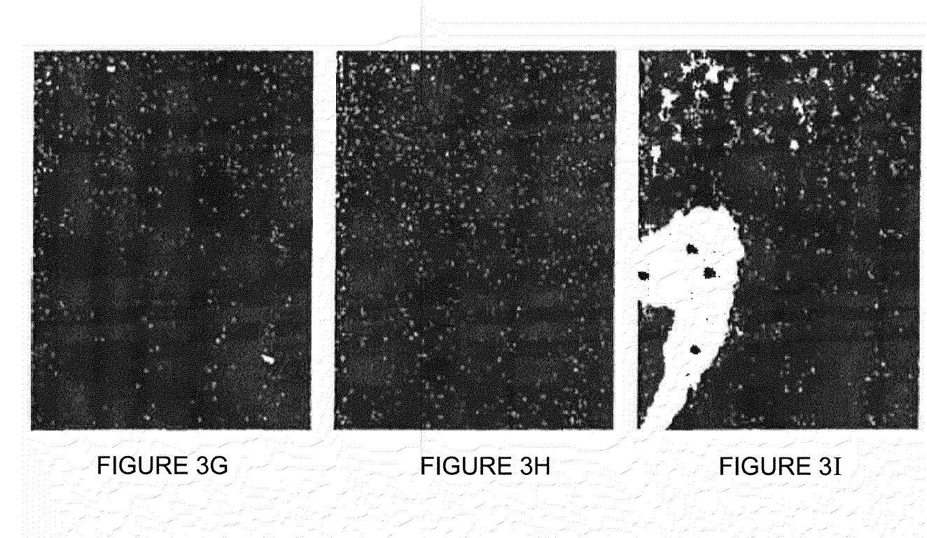

TAP-1 expression after infection of B16F10 cells with AdhTAP-1 increases the expression of overall surface MHC Class I levels (FIG. 2) and is able to resurrect the presentation of the TRP-2 peptide on H-2K^{b} to allow TRP-2-specific CTL killing (FIG. 3). Stronger responses are seen in a human TAP-deficient melanoma cell line, buf1280. Since these cells are much more sensitive to human adenovirus infections than mouse cells, a much lower multiplicity of infection is needed to express hTAP-1 compared to B16F10. Surface levels of both endogenous HLA-ABC and transgene-expressed H-2K^{b} are increased in buf1280 cells infected with AdhTAP-1, and higher susceptibility to killing upon viral superinfection is also observed in both these cell lines. These data show that adenovirus-expressed hTAP-1 resurrects the otherwise-deficient MHC Class I antigen processing pathway in these cells.

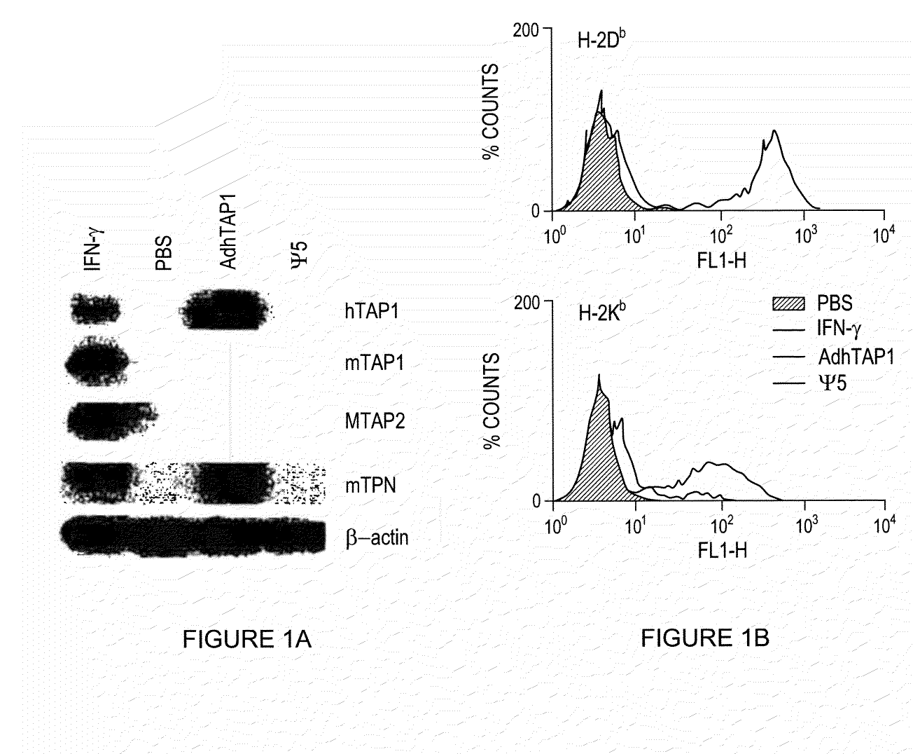

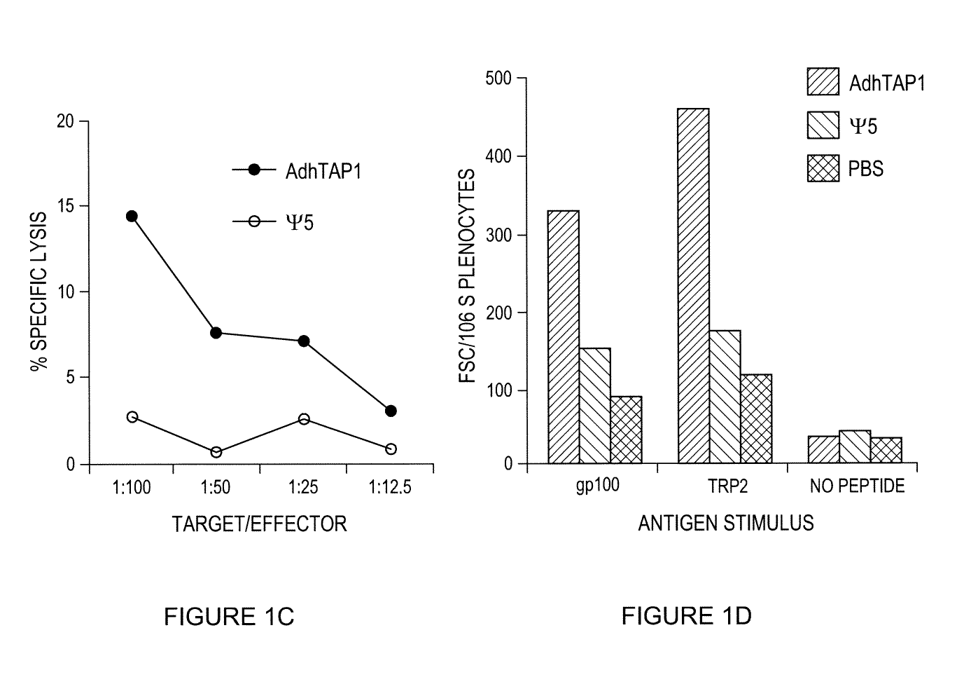

It is also surprising that adenovirus-driven hTAP-1 expression in B16F10 cells and in CMT.64 lung carcinoma cells greatly increased endogenous mouse tapasin expression (FIG. 1). Providing a source of hTAP-1 therefore appears to restore this additional component of the classical antigen presentation pathway, either by stabilization or by inducing its expression, thereby restoring the pathway needed to present TAA on MHC Class I at the cell surface. Interestingly, however, an increase in endogenous murine TAP-2 expression is not observed. Therefore at present it is unknown how TAP-1 alone transports peptides into the ER. A recent study has shown that preexisting TAP-1 is necessary for stable protein expression of TAP-2, therefore high levels of TAP-1 expressed from the transgene may stabilize very low levels of TAP-2 in the tumor cells and allow the formation of small numbers of functional heterodimers. In conjunction with restored tapasin levels, these transporters may allow the import of TAA-derived peptides and loading onto MHC I. However, increased TAP-2 protein expression in AdhTAP-1-infected tumor cells has not been detected.

TAP, and associated components of the peptide-loading complex, are not only essential for direct antigen presentation to CD8⁺T cells, but are also required for the cross-presentation of exogenous antigens to CD8⁺T cells in order to initiate immune responses to tumors. It is clear that vaccination of mice with AdhTAP-1 results in immunologically-mediated anti-tumor responses. AdhTAP-1 treatment results in a significant reduction of tumor mass in mice bearing mouse melanoma B16F10 when compared to those treated with vector alone and PBS controls.

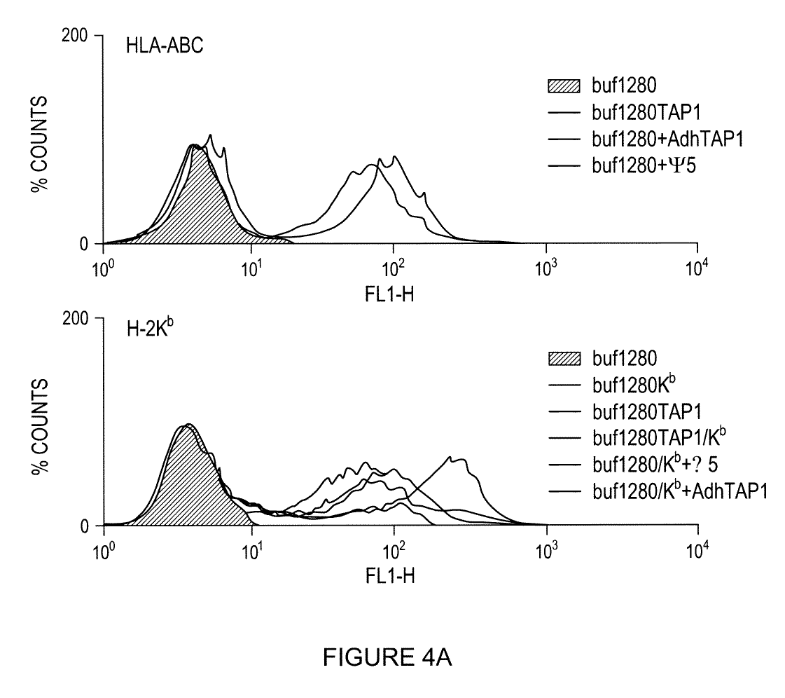

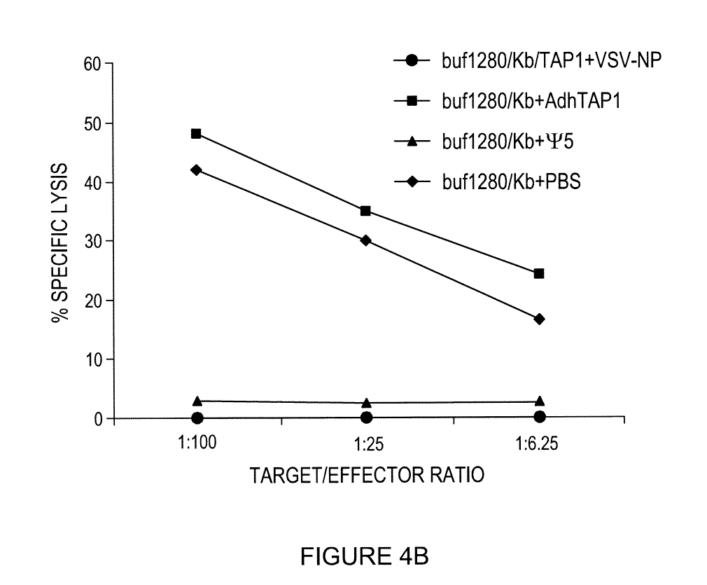

Immunohistochemical analysis shows a significant increase in CD4+ and CD8+ T cells and CD11c⁺ DCs within tumors of mice treated with AdhTAP-1, and flow cytometric analysis also reveals increased CD8+ T cell levels in mice treated with AdhTAP-1 compared to PBS and controls. Interestingly, CD4⁺ levels appear to be increased in tumors from both AdhTAP-1 and treated mice, indicating the viral vector alone may promote increased CD4⁺ T cell responses. In addition, TAA-specific (including gp100 and TRP-2) IFN-γ-secreting splenocytes are observed after vaccination with irradiated B16F10 cells infected with AdhTAP-1, indicating that TAP activity in tumor cells can promote Th1 responses (FIG. 4). It has been demonstrated that infection with AdhTA-1 significantly increased the cross-presentation of the acquired antigen ovalbumin by H-2K^{b} in splenic DCs. TAP-1 activity or the products of TAP-1 activity in AdhTAP-1-infected B16F10 cells must therefore be transferred in some way to the DCs involved in the cross-presentation of MHC class I antigens. Perhaps TAP-I expression in B16F10 melanoma tumor cells results in increased expression of a variety of antigens that may be bound to MHC class I on the surface of these cells, and these MHC class I-restricted antigens may be transferred to MHC Class I on DCs. Alternatively, DCs possibly access processed tumor antigens from the ER (endoreticulum) compartment of B16F10 cells during internalization and antigen cross-presentation.

Among the most striking attributes of adaptive immunity is the phenomenon of immunological memory. Both CD8⁺ and CD4⁺ T memory cells are critical for long-term cancer vaccine efficacy. It has been found that AdhTAP-1 increased the numbers of both CD4⁺ and CD8⁺ memory cells (CD43^{lo}CD44^{hi}CDI27^{hi}CD8⁺ and CD43^{lo}CD44^{hi}CD127^{hi}CD4⁺ phenotypes) in B16F10 tumor-bearing when mice compared with vector Ψ5 and PBS control groups, indicating that TAP-1 therapy may be particularly well-suited to promoting long term preventative or therapeutic immune responses to tumors. Furthermore, efficacy is achieved by injection of just 1×10⁸ pfu of non-replicating virus per mouse. On average, adult mice contain 1×10¹¹ normal cells and in our experiments 1.5×10⁵ transplanted autologous metastatic and disseminated tumor cells. Although at most only 1 in 1000 cells are infected by the non-replicating virus, tumor growth appears to be inhibited by promoting a protective immune response that after priming is able to recognize even the smallest amount of neoantigen/MHC I complex at the cell surface of the tumors. These results suggest that TAP treatment should be considered for use in cancer immunotherapies as it is independent of the HLA type of the host and the antigenic complement of the tumor, promotes T cell memory, and has efficacy even when not all of the tumor cells are infected by the viral delivery vector.

A highly attractive approach to cancer immunotherapy is the re-introduction of TAP-1 in TAP-deficient cancers, since TAP-1 is not only essential for direct antigen presentation to CD8⁺ T cells, but is also required for the cross-presentation of exogenous antigen in dendritic cells during the initiation of the immune response. TAP loss is common in melanoma patients. The murine B16 melanoma system represents an important in vivo model for the evaluation of T cell-based immunization and vaccination strategies. B16F10 cells, established from a C57BL/6 melanoma, were established after successive selections for lung metastases after intravenous injection, and are highly metastatic. B16F10 cells are poorly immunogenic, not surprisingly since they express no MHC class II and very low levels of MHC class I, although both are inducible upon treatment with IFN-γ. They are defective in many components of the antigen processing and presentation pathway, including TAP-1 and TAP-2, LMP-2, 7, and 10, PA28a and B, and tapasin (Tpn). Interestingly, all of these defects can also be corrected by IFN-γ treatment.

The invention is further described and illustrated in the following examples which are not intended to limit the specifically enumerated embodiments or the scope of the appended claims. The pertinent portions of all cited references are incorporated herein in their entirety.

### Materials and Methods

As an approximation, a 20 gram mouse contains 1×10¹¹ cells. Therefore, in this study we examine the effect of inoculating 1×10⁸ pfu of non-replicating adenovirus containing TAP-1 (1/1000 of the total cells in the animal), in order to determine whether we can achieve an efficacious outcome against the transplanted autologous, MHC I-deficient, B16F10 melanoma cells.

### Mice

Female, 6- to 8-week-old C57BL/6 (H-2^{b}) mice were obtained from The Jackson Laboratory (Bar Harbor, Me.) and housed and bred at the Biotechnology Breeding Facility, University of British Columbia, according to the guidelines of the Canadian Council on Animal Care and the University of British Columbia Animal Care Committee.

### Cells and Viruses

Human cell lines 293 (ATCC, CRL-1573, Rockville, Md., USA), Ti (ATCC, CRL-1991, TAP positive) and T2 (ATCC, CRL-1992, TAP negative) were cultured in Dulbecco's modified Eagles medium supplemented with 10% fetal bovine serum (except T2 cells, which received 20% fetal bovine serum), 200 mM L-glutamine, streptomycin (0.1 mg/ml), and penicillin (100 u/ml). Murine tumor cell lines RMA-3 (TAP deficient, a gift from Dr. Peter Cresswell), B16F10 cells (ATCC, CRL-6475, TAP deficient), human melanoma buf1280 cells (TAP deficient) and buf1280 stably transfected with human TAP-1 (buf1280 TAP-1) (both kindly provided by Dr. Barbara Seliger) were cultured in RPMI medium with the same supplements. The virus Ψ5 is an E1 and E3 deleted version of adenovirus (Ad5) containing loxP sites flanking the packaging site. Non-replicating adenovirus encoding hTAP-1 under the control of human CMV immediate-early promoter (AdhTAP-1) has been previously described in detail. The Ψ5 and AdhTAP-1 viruses were propagated and titrated in 293 cells. Splenocytes were cultured in complete culture medium consisting of RPMI 1640, 2 mM L-glutamine, 1% penicillin/streptomycin, 50 μM β-mercaptoethanol, 1 mM sodium pyruvate, 0.1 mM essential amino acids and 10% fetal bovine serum.

### Stable Transfection

A plasmid encoding the entire mouse H-2K^{b} gene, pMX-pie/K^{b} (made in our laboratory) was transfected into 80% confluent monolayers of buf1280 and buf1280TAP-1 cells using the lipofectAMINE PLUS™ reagent protocol (Invitrogen Life Technologies, Carlsbad, Calif.). Buf1280 and buf1280 TAP-1 cells expressing H-2K^{b} were selected in medium supplemented with puromycin (2 μg/ml), and H-2K^{b} expression was confirmed by flow cytometric analysis.

### Synthetic Peptides

Vesicular stomatitis virus nucleoprotein (VSV)-NP52-59 peptide (RGYVYQGL) and the B16F10 TAAs, tyrosinase-related protein-2 (TRP-2)₁₈₀₋₁₈₈ (VYDFFVWL) and gp100₂₅₋₃₃ (KVPRNQDWL), were made by the Peptide Synthesis Lab at the University of British Columbia. The purity of peptides was determined by HPLC to be >95% and the identities were confirmed by mass spectrometry. Lyophilized peptides were dissolved in DMSO at 10 mg/ml.

Measurement of TAP and tapasin expression after AdhTAP-1 infection of B16F10 cells B16F10 cells growing as a monolayer were infected with AdhTAP-1 or Ψ5 at 50 pfu/cell. T1 cells and T2 cells were respectively used as positive and negative controls for hTAP-1 expression. B16F10 cells treated with IFN-γ and untreated B16F10 cells were positive and negative controls, respectively, for mouse TAP-1 (mTAP-1), mouse TAP-2 (mTAP-2) and mouse tapasin (mTpn) expression. Two days after infection, the cells were washed with Tris saline (10 mM Tris HCl, pH 7.4, 120 mM NaCl) and extracted on ice for 50 min in RIPA buffer containing 1% Nonidet P-40, 1% sodium deoxycholate, 0.1% SOS, 1 mM phenylmethylsulfonylfluoride, and aprotinin (1 μg/ml) and a 1:100 dilution of protease inhibitor cocktail (Sigma, Saint Louis, Mo.). Cell extracts were clarified by centrifugation 12,000 g at 4° C. for 15 min. The samples were subjected to 50S-PAGE, electrotransferred to nitrocellulose and probed with rabbit anti-hTAP-1 antiserum with no cross-reactivity to mouse TAP-1 (Stressgen Biotechnologies Corp, Victoria, BC, Canada), rabbit anti-hTAP-1 antiserum (a gift from Dr. David Williams, University of Toronto), rabbit anti-mTAP-1 and rabbit anti-mTAP-2 antisera (made in our lab by immunizing rabbits with the TAPI peptide sequence RGGCYRAMVEALAAPAD-C, and the last 16 C terminal amino acids of TAP-2, respectively, coupled via the C-terminal cysteine to KLH carrier (Pierce Biotechnology Inc, Rockford, Ill.) and tested by Western blotting with fibroblasts from TAP-1-expressing and TAP-1-deficient mice), and mouse monoclonal anti-β-actin antibody (Sigma-Aldrich, Oakville, ON, Canada). The second antibodies were goat anti-rabbit IgG (H+L)-HRP and goat anti-mouse IgG (H+L)-HRP (Jackson ImmunoResearch Lab, West Grove, Pa.). Immunoreactive protein bands were visualized by exposure to Hyperfilm (Amersham Biosciences, Little Chalfont, Buckinghamshire, England) using an enhanced chemiluminescence (ECL) detection system (Amersham Biosciences).

### Measurement of Surface Expression of MHC Class I by Flow Cytometric Analysis

B16F10, buf1280, buf1280 TAP-1, buf1280/K^{b}, and buf1280 TAP-1/K^{b} cells were infected with AdhTAP-1 or Ψ5 at 50 PFU/cell (for B16F10 cells), or 3 PFU/cell (for buf1280, buf1280 TAP-1, buf1280/K^{b}, and buf1280 TAP-1/K^{b} cells). Forty-eight hours after infection, the B16FI0, buf1280/K^{b}, and buf1280 TAP1/K^{b} cells were incubated with anti-MHC class monoclonal antibodies, y3 (H-2K^{b}-specific, ATCC) and 28.14.8S (H-2K^{b}-specific, ATCC) at 4° C. for 30 min. After three washes with PBS, the bound antibodies were detected using goat anti-mouse IgG-FITC (Jackson ImmunoResearch Lab). Buf1280, and buf1280 TAP-1 were incubated with anti-HLA ABC-PE antibodies (BD Phannagen, Mississauga, ON, Canada) at 4° C. for 30 min. The flow cytometric analysis was performed using a FACSCalibur™® (Becton Dickinson, Franklin Lakes, N.J.) flow cytometer.

### Generation of VSV and TRP-2 Specific Effector T Cells

H-2K^{b}-restricted, VSV-specific CTL effectors were generated by intraperitoneal (i.p.) injection n of 5×10⁷ pfu of VSV into mice. Splenocytes were collected five days after infection and cultured in complete RPMI-1640 medium containing 1 μM VSV-NP₅₂₋₅₉ for five days. To generate H-2K^{b}-restricted TRP-2-specific CTLs, 100 μg TRP-2₁₈₀₋₁₈₈ were mixed with 50 μl TiterMax adjuvant (CedarLane Laboratories Ltd, Hornby, ON, Canada) and 50 μl PBS and injected subcutaneously (s.c.) into mice. This procedure was repeated after 7 days. Fourteen days after the initial injection, mice received an additional injection (i.p.) with TRP-2 peptide-pulsed, irradiated RMA-S cells (5×10⁶ cells in 300 μl). The RMA-S cells were prepared by incubating 5×10⁶ cells with TRP-2₁₈₀₋₁₈₈ peptide (10 μg/ml peptide in 2 ml medium) overnight at room temperature followed by γ-irradiation (10,000 rads). The cells were washed with PBS and re-suspended in 300 μl PBS. Seventeen days after the initial injection, the immunized spleen was removed and 10⁸ splenocytes were cultured for five days with 5×10⁷ γ-irradiated naive splenocytes pulsed with TRP-2 peptide (10 μl/ml).

### ⁵¹Cr-Release Cytotoxicity Assay

A standard 4 hr ⁵¹Cr-release cytotoxicity assay was used to measure CTL activity against target cells. For VSV-specific killing, buf1280, buf1280 TAP-1, buf1280/K^{b}, and buf1280 TAPI/K^{b} were infected with AdhTAP-1 or Ψ5 at 3 PFU/cell for 1 day and super-infected with VSV at 10 pfu/cell overnight and used as targets. For TRP-2₁₈₀₋₁₈₈ specific killing, B16F1O cells were infected with AdhTAP-1 or Ψ5 at 50 pfu/cell or mock infected with PBS. All targets were labeled with ⁵¹Cr by incubating 10⁶ cells with 100 μCi of ⁵¹Cr (as sodium chromate; Amersham, Arling Heights, Ill.) in 250 μl of complete RPMI medium for 1 hr at 37° C., washed three times with PBS, then incubated with the effector cells at the indicated killer:target ratios for 4 hrs. One hundred microlitres of supernatant were collected from each well and the percentage of ⁵¹Cr release was calculated using the formula: % release=100×(cpm experiment−cpm spontaneous release)/(cpm maximum release−cpm spontaneous release).

### ELISPOT Assay

To generate B16F10 antigen specific splenocytes, 6×10⁶ B16F10 cells were incubated with AdhTAP-1 or Ψ5 at 50 PFU/cell or PBS at 37° C. for 2 hrs. After incubation, the cells were irradiated (10,000 rad) for 30 min, then washed and re-suspended in PBS. On days 1, 4, and 8, mice were immunized by three separate intraperitoneal (i.p.) injections of 2×10⁶ irradiated cells (three mice per group). Nine days after the last immunization the spleens from each group were pooled and their splenocytes were isolated and cultured in vitro in complete RPMI-1640 medium containing B16F10 tumor associated antigen peptide TRP-2₁₈₀₋₁₈₈ or gp100₂₅₋₃₃ (20 ug/ml) for 14 hours. Controls contained no peptide. The frequency of B16F10 TAA-specific IFN-γ secreting cells was determined using an ELISPOT assay (R&D Systems, Minneapolis, Minn.) according to the manufacturer's instructions. Briefly, dilutions of splenocytes ranging from 1×10⁶ to 1×10⁴ cells/well in 100 μl of complete RPMI-1640 medium were transferred to duplicate wells, with either TRP-2₁₈₀₋₁₈₈ orgp100₂₅₋₃₃ peptide (2 μg/ml) or without peptide. Following overnight incubation at 37 C in 5% C0₂ in air, the cells were removed, and the wells washed and incubated with biotinylated rabbit anti-IFN-γ antibody. After further washing, bound anti-IFN-γ antibody was detected with alkaline phosphatase conjugated streptavidin. Spots were developed by incubating the plate with the chromogen BCIP/NBT. The color reaction was stopped by washing with deionized water. The plates were air-dried, and spots were visualized and counted using a dissecting microscope.

Treatment of BI6F10 Tumor Bearing Mice with AdhTAP-1

Twelve mice in each group were injected subcutaneously (s.c.) with 1.5×10⁵ B16F10 cells in 100 μl PBS). On days 1, 4, and 8 after the introduction of BI6F10 cells, mice were injected s.c. with either AdhTAP-1, Ψ5, or PBS 1×10⁸ pfu/mouse/injection in 100 μl PBS). Fifteen days after the introduction of tumor cells, the mice were killed and their tumor masses were measured. One tumor from each group was frozen for immunohistochemical (IHC) staining. The remaining tumors were pooled to measure the number of tumor infiltrating lymphocytes by flow cytometric analysis. In addition, the spleens were pooled and single-cell suspensions were prepared prior to measurement of memory T cells by flow cytometry (see below).

### Phenotyping of Memory T Cells

Spleens were washed and single cell suspensions prepared by gentle teasing. After counting, cells were incubated with anti-CD4 (L3T4)-FITC or anti-CD8α anti-(Ly-2)-FITC, anti-CD43-PE (1B11), anti-CD44-APC (pgp-1) and anti-CD127 (B 12-1)-Biotin monoclonal antibodies on ice for 50 min and then with streptavidin-PerCP-cy5.5 (all the reagents were from BD-Pharmingen, Mississauga, ON, Canada) for 35 min. Cells were then analyzed by flow cytometry (50,000 events/sample).

### Measurement of Tumor Infiltrating Cells

T cells (CD4⁺ and CD8⁺) and dendritic cells (DC; CD11c⁺) were analyzed by flow cytometry and visualized in situ by immunohistochemistry. For the detection of tumor infiltrating lymphocyte subsets (CD4⁺ and CD8⁺ T cells), tumors were washed extensively and single cell suspensions prepared and filtered through 40 μM nylon filters. Live cells were isolated from debris and dead cells using density centrifugation with Ficoll-Paque™ PLUS (Amersham Biosciences), and were incubated with rat anti-mouse CD8α (Ly-2)-FITC and rat anti-mouse CD4 (L3T4)-PE monoclonal antibodies and 7AAD (Molecular Probes, Eugene, Oreg.) prior to flow cytometric analysis. Dead cells, (86% of the cell population) detected by staining with 7AAD, were gated out and the remaining 14% of the cells were assessed for CD4 and CD8 expression. For immunohistochemical staining of tumor infiltrating cells (CD8⁺ and CD4⁺ T cells, and CD4⁺ T cells, and CD11c⁺ DCs), frozen tumors were sectioned (8 nm) and acetone-fixed following standard procedures. Sections were incubated with rat anti-mouse CD4 monoclonal antibody (RM4-5), rat anti-mouse CD8 monoclonal antibody, and hamster anti-mouse CD11c monoclonal antibody (HL3). Rat IgG₂ₐ and hamster IgG were used as isotype controls. Antibody binding was detected with biotinylated polyclonal anti-rat IgGs and biotinylated anti-hamster IgG cocktail secondary antibodies and streptravidin-HRP (DAB detection system). All the reagents were purchased from BD-PharMingen.

### Statistics

The statistics for the in vivo tumor studies were performed using a paired t-test, The data were considered statistically different if p<0.05.

### EXAMPLES

### TAP-1 Expression in AdhTAP-1-Infected B16F10 Melanoma Cells

Immunoblot analysis showed that infection of B16F10 cells with AdhTAP-1 resulted in high levels of expression of the transgene hTAP-1. In addition, hTAP-1 expression in AdhTAP-1-infected B16F10 cells increased endogenous mTpn expression, but not TAP-1 and TAP-2 expression (FIG. 1A).

MHC class I Surface Expression in AdhTAP-1-Infected B16F10 Cells is Increased

We investigated the effect of increased hTAP-1 expression on MHC class I surface expression in AdhTAP-1-infected B16F10 cells by flow cytometry, The results showed that the cell surface expression of both H2-K^{b} and H2-D^{b} was significantly increased in B16F10 cells infected with AdhTAP-1 when compared with uninfected cells or, cells infected with the vector Ψ′5 alone. The expression of TAP-1 alone at least partially restored MHC class I expression on the surface of B16F10 cells when compared to MHC class I expression on the surface of 1FN-γ treated cells (FIG. 1B).

### Antigenicity of AdhTAP-1-Infected B16F10 Cells is Restored

Cytotoxicity assays were performed in order to test whether the AdhTAP-1-induced MHC class I expression enhanced the ability of B16F10 cells to present the H-2K_{b}-restricted tumor associated antigen (TAA), TRP-2_{180. 188}. The results showed that the AdhTAP-1-infected B16F10 cells were sensitive to the cytolytic activity of the TRP-2_{180. 188}-specific effectors, while the uninfected B16F10 cells or the Ψ′5-infected cells (negative controls) were resistant to killing (FIG. 1C). This indicates that TAP-1 expression and activity by AdhTAP-1 infection can restore sufficient MHC class I-restricted antigen presentation of a TAA, TRP-2₁₈₀₋₁₈₈, to render these cells susceptible to killing by specific cytotoxic lymphocytes.

### AdhTAP1 Increases TAA-Specific IFN-γ-Secreting Splenocytes

TRP-2₁₈₀₋₁₈₈ and gp100₂₅₋₃₃ specific cellular immune responses elicited by the AdhTAP-1-infected B16F10 cells were measured by IFN-ELISPOT assay. TRP-2 and gp100 are known differentiation antigens expressed in B16F10 cells and other melanoma cells from human patients. Mice that were vaccinated with irradiated, AdhTAP-1-infected B16F11 cells showed a significant increase in the number of both TRP-2₁₈₀₋₁₈₈- and gp100₂₅₋₃₃-specific, IFN-γ-secreting splenocytes when compared to those vaccinated with either irradiated uninfected cells or irradiated Ψ5-infected B16F10 cells. These results indicate that AdhTAP-1 infection of B16F10 cells induced a Th1-type tumor specific immune response (FIG. 1D).

### AdhTAP-1 Inhibited Tumor Growth in B16F10 Tumor-Bearing Mice and Increased Tumor-Infiltrating Lymphocytes and Memory Cell

We examined whether AdhTAP-1 infection of B16F10 cells inhibited tumor formation in mice. The data showed that five (100%) of the B16F10 tumor-bearing mice treated with AdhTAP-1 were tumor-free, in comparison with one and two tumor-free mice respectively in the Ψ5 and PBS-treated groups. The mean tumor weight in tumor-bearing mice treated with AdhTAP-1 was 16 mg, in sharp contrast to the 152 mg and 157 mg means of the Ψ5 and PBS-treated groups, respectively. AdhTAP-1 treatment of tumor-bearing mice therefore significantly reduced the tumor mass in comparison to those treated with and Ψ5 and PBS (p<0.01, FIG. 2C).

One tumor from each group of mice was examined for infiltrating CD4⁺, CD8⁺ T cells and CD11c⁺ DCs by immunohistochemistry. The results showed that mice treated with AdhTAP-1 had significantly greater numbers of CD4⁺ and CD8⁺ T cells and CD11c⁺ DCs in the tumor masses when compared to tumors taken from mice treated with PBS or Ψ5 (FIG. 3). In addition, tumors harvested from the rest of each group of mice were pooled and examined for CD4⁺, CD8⁺ T cells by flow cytometry. Mice treated with AdhTAP-1 had a significantly higher percentage of CD8⁺ T cells in their tumors when compared with tumors taken from mice treated with PBS or Ψ5. In addition, tumors from mice treated with AdhTAP1 or Ψ5 had increased CD4⁺ T cell levels when compared to tumors from mice treated with PBS (FIG. 3).

Splenocytes from mice infected with AdhTAP-1, Ψ5 or PBS were also investigated for their levels of memory T cells by flow cytometry. The results showed that mice treated with AdhTAP-1 had significantly greater numbers of memory T cells (CD43^{lo}CD44^{hi}CDI27^{hi}CD8⁺ and CD43^{lo}CD44^{hi}CDI27^{hi}CD4⁺) per spleen when compared with the mice treated with PBS or Ψ5. Mice treated with AdhTAP-1 had 1.8×10⁷ CD43^{lo}CD44^{hi}CD127^{hi} CD4⁺ and 1.6×10⁷ CD43^{lo}CD44^{hi}CD127^{hi}CD8⁺T cells per spleen compared to 1.1×10⁷ and 1.0×10⁷ per spleen for Ψ5 controls, and 9.0×10⁶ and 8.0×10⁶ for PBS controls, respectively.

### AdhTAP-1 Restores Antigen Presentation in Human Melanoma Cells

Mouse melanoma cells such as B16F10 are not a very sensitive model for AdhTAP-1 infection since the normal host cells for human adenoviruses are human cells, not murine cells.

Therefore, we tested whether or not AdhTAP-1 induced HLA surface expression and antigenicity in a more sensitive model, the human melanoma cell line, buf1280. As expected, AdhTAP-1 infection at as low as 3 pfu/cell greatly increased surface HLA-ABC expression in buf1280 cells (FIG. 4a) and H-2K^{b} expression in buf1280/K^{b} cells which stably express H-2K^{b} (FIG. 4b). In contrast, infectious doses as high as 50 pfu/cell of AdhTAP-1 were required to induce surface H-2K^{b} expression in B16F10 cells (FIG. 1B). AdhTAP-1 infection increased MHC class I surface expression in buf1280 and buf1280/K^{b} cells to even higher levels than those seen in buf1280 TAP-1 and buf1280 TAP-1-K^{b} cells stably expressing hTAP-1 (FIGS. 4a and 4b).

A cytotoxicity assay was used to determine if AdhTAP-1-induced MHC class I surface expression enhanced the capability of buf1280-K^{b} cells to present antigens. Stably transfected buf1280/K^{b} cells infected with AdhTAP-1 or Ψ5 and superinfected with Vesicular Stomatitis Virus (VSV) were used as targets for VSV-specific effectors. The results showed that buf1280/K^{b} cells infected with AdhTAP-1 at as low as 3 pfu/cell (and super-infected with VSV) and buf1280 TAP1/K^{b} infected only with VSV (positive control) were sensitive to the cytolytic activity of the VSV-specific effectors. In contrast, buf1280 and buf1280/K^{b} cells infected with Ψ5 and VSV, buf1280 cells super-infected with AdhTAP-1 and VSV and buf1280 TAP-1 cells infected only with VSV (all negative controls) were resistant to killing. AdhTAP-1 induced even greater killing of buf1280/K^{b} cells when compared with buf1280 TAP1/K^{b} cells stably expressing hTAP-1, presumably due to higher expression of hTAP-1 from the adenovirus. These results show that TAP-1 expression and activity caused by AdhTAP-1 infection of human melanoma cells at low doses can restore sufficient MHC class I-restricted antigen presentation of a specific epitope (VSV-NP) to render these cells susceptible to specific cytotoxic activity.

What is claimed is:

1. A method of inducing protective immunity in animals against TAP-deficient metastatic melanoma by treating the animals with relatively small amounts of a recombinant non-replicating adenovirus encoding TAP-1.

2. The method of claim 1 wherein the animal is a human.

3. The method of claim 1 wherein the adenovirus only infects a small fraction of the metastatic tumor cells.

4. The method of claim 1 wherein the amount of recombinant adenovirus used is in the range of from about 1×10⁹ to 1×10⁷ cells per inoculum.

5. The method of claim 1 wherein the adenovirus encoding TAP-1 is part of a viral vector.

6. The method of claim 1 wherein the viral vector is a VSV vector.
